(19) United States
(12) Patent Application Publication
(10) Pub. No.: US 2004/0035431 A1
(43) Pub. Date: Feb. 26, 2004

(54) Ear cannula system and method of using same

(76) Inventor: Clifford A. Wright, San Diego, CA (US)

Correspondence Address: JERRY RICHARD POTTS, 3248 VIA RIBERA, ESCONDIDO, CA, 92029, US

(21) Appl. No.: 10/634,365

(22) Filed: Aug. 5, 2003

Related U.S. Application Data
(63) Continuation-in-part of application No. 10/224,849, filed on Aug. 21, 2002.

(51) Int. Cl. A61M 15/08

(52) U.S. Cl. 128/207.18

(57) ABSTRACT

A fluid delivery system includes a stop disposed at a distal end of one of a pair of slider extension tubes, the one of a pair of slider extension tubes being slidingly received within a tube channel of one of a pair of elongated flexible ear pieces for facilitating supporting therefrom a nasal cannula and for facilitating adjusting the distance between the nasal cannula and the one of a pair of elongated flexible ear pieces. The other one of a pair of slider extension tubes being slidingly received with another tube channel disposed in the other one of a pair of elongated flexible ear pieces for facilitating supporting therefrom the nasal cannula and for facilitating adjusting the distance between the nasal cannula and the other one of a pair of elongated flexible ear pieces and the nasal cannula. The other one of a pair of slider extension tubes having its distal end coupled to a section of oxygen tubing having a oxygen source connector disposed on its distal end and a plastic securing clip mounted thereto for facilitating securing the oxygen tubing to a stationary item.

### RELATED APPLICATIONS

This is a continuation-in-part of U.S. patent application Ser. No. 10/224/849 Entitled Oxygen Delivery System and Method of Using Same, filed Aug. 21, 2002, now U.S. Patent No. ______.

### FIELD OF THE INVENTION

This invention relates in general to oxygen delivery systems, and more particularly to an oxygen delivery system that includes a nasal cannula assembly having ear pieces and extension tubes for securing the nasal cannula at a desired position on a user.

### BACKGROUND

Oxygen delivery systems that include nasal cannulas are well known in the art. Examples of such prior art systems include U.S. Pat. Nos. 6,328,038B1; 6,298,850; 5,682,881; 5,636,630; 5,438,979; 5,271,391; 5,117,818; 5,025,805; 4,836,200; 4,808,160; 4,753,233; 4,739,757; 4,699,139; 4,422,456; 4,406,283; 4,156,426; 4,106,505; 3,802,431; 2,868,199; 2,763,263; and 2,168,705.

While nasal cannulas are a convenient method of supplying a patient with oxygen enriched gases, it would be highly desirable to have a new and improved oxygen delivery system that includes a nasal cannula that is easily adjusted for the comfort of the patient and that is not prone to falling off the face of the patient.

### SUMMARY OF THE INVENTION

An oxygen-delivery system includes a nasal cannula having a pair of nasal prongs that may be sized in length for insertion into the nasal cavities of a user. The nasal cannula is coupled at one of its ends to a right side extension tube that passes through an elongated flexible earpiece that is adapted to be hooked over one ear of the user. The right side extension tube is plugged at its distal end. The other end of the nasal cannula is coupled to a left side extension tube that passes through another elongated flexible earpiece that is adapted to be hooked over the other ear of the user. The distal end of the left side extension tube is coupled to an oxygen tube having a plastic mounting clip attached thereto to permit the oxygen tube to be secured to a fixed object such as the shirt of the user or a bed sheet should the user be confined to a bed. The ear pieces cooperate with the extension tubes to allow the pieces to be adjusted to fit over the ears of the user with the nasal cannula positioned in the nasal passageways of the user and with the extension tubes exit to the rear of the user thereby providing a secure, snug fit without otherwise interfering with the face or neck of the user. According to the method of using the oxygen delivery system a user pieces the distal end ear piece over one ear, inserts the nasal prongs into his or her nasal cavities, and moves the ear piece relative to the extension tube to an adjusted position that allows the other ear piece to be likewise adjusted so it can be securely hooked over the other ear of the user with the nasal prongs of the nasal cannula comfortably inserted into the nasal cavities of the user.

### BRIEF DESCRIPTION OF THE DRAWINGS

FIG. 1 depicts a frontal view of a patient's head showing a typical prior art nasal cannula arrangement for delivering oxygen to a patient;

FIG. 2 depicts a perspective view of a patient using a preferred embodiment of the present invention;

FIG. 3 depicts an ear cannula system, which is constructed in accordance with the present invention;

FIG. 4 depicts an adjustment step in the novel method of using the oxygen system;

FIG. 5 is a bottom view of a plastic clip forming part of the ear cannula system of FIG. 1;

FIG. 6 is a left side plane view of the plastic clip forming part of the ear cannula system of FIG. 1;

FIG. 7 is a front plane view of the plastic clip forming part of the ear cannula system of FIG. 1;

FIG. 8 is a right side plane view of the plastic clip forming part of the ear cannula system of FIG. 1;

FIG. 9 is a top plane view of the plastic clip forming part of the ear cannula system of FIG. 1;

FIG. 10 is a pictorial view of the plastic clip forming part of the ear cannula system of FIG. 1;

FIG. 11 is a bottom plane view of a mounting strap forming part of the ear cannula system of FIG. 1;

FIG. 12 is a left side plane view of the mounting strap forming part of the ear cannula system of FIG. 1;

FIG. 13 is a top plane view of the mounting strap forming part of the ear cannula system of FIG. 1;

FIG. 14 is a right side plane view of the mounting strap forming part of the ear cannula system of FIG. 1;

FIG. 15 is a section view of the mounting strap taken along line 15-15 of FIG. 13;

FIG. 16 is a pictorial view of the mounting strap forming part of the ear cannula system of FIG. 1;

FIG. 17 is a front plane view of an ear piece forming part of the ear cannula system of FIG. 1;

FIG. 18 is a side plane view of the ear piece forming part of the ear cannula system of FIG. 1;

FIG. 19 is a section view of the ear piece taken along line 19-19 of FIG. 18;

FIG. 20 is a top plane view of the ear piece forming part of the ear cannula system of FIG. 1; and

FIG. 21 is a pictorial view of the ear piece forming part of the ear cannula system of FIG. 1.

### DETAILED DESCRIPTION

Before discussing the preferred embodiment of the present invention, it may be beneficial to briefly review a typical prior art oxygen delivery system 510 that utilizes a nasal cannula 512. In this regard, as best seen in FIG. 1, the nasal cannula 512 generally comprises a nosepiece or nasal assembly 514 having a hollow body member 516 with two upstanding nose prongs or nasal extension tubes 518 and 520 that are adapted to be placed in the nasal cavities of the patient P. Oxygen (from a source not shown) is supplied to the hollow body member 516 at ones of its end openings allowing the body member 516 to functions as a gas distribution manifold. Generally, a pair of gas supply tubes 522 and 524 are attached to the nosepiece 514, that is supported or held in place by extending the gas supply tubes 522 and 524 from the nosepiece 514 to respective ones of the ears of the patent P so the tubes 522 and 524 pass behind respective ones the ears of the patient P. The extension tubes 522 and 524 are bent downward behind the ears and traverse along the jaw area and are then secured together by a cinch 526 or an adjustable loop that is tightened below the chin of the patient to hold the nosepiece in place. The tubes are then joined in by a reducer (not shown) so that a single gas line is available to be attached to the oxygen or air source. From the foregoing, it should be understood that the looping tubes that extend around the ears of the user and along the jaw area of the patient and down to the neck area are uncomfortable and can be dislodged if the cinch 526 is not properly adjusted to tighten the loops sufficiently around the ears of the patient P in a somewhat uncomfortable manner.

Therefore the is a need for a new and improved oxygen delivery system that includes a nasal cannula that is easily adjusted for the comfort of the patient and that is not prone to falling off the face of the patient.

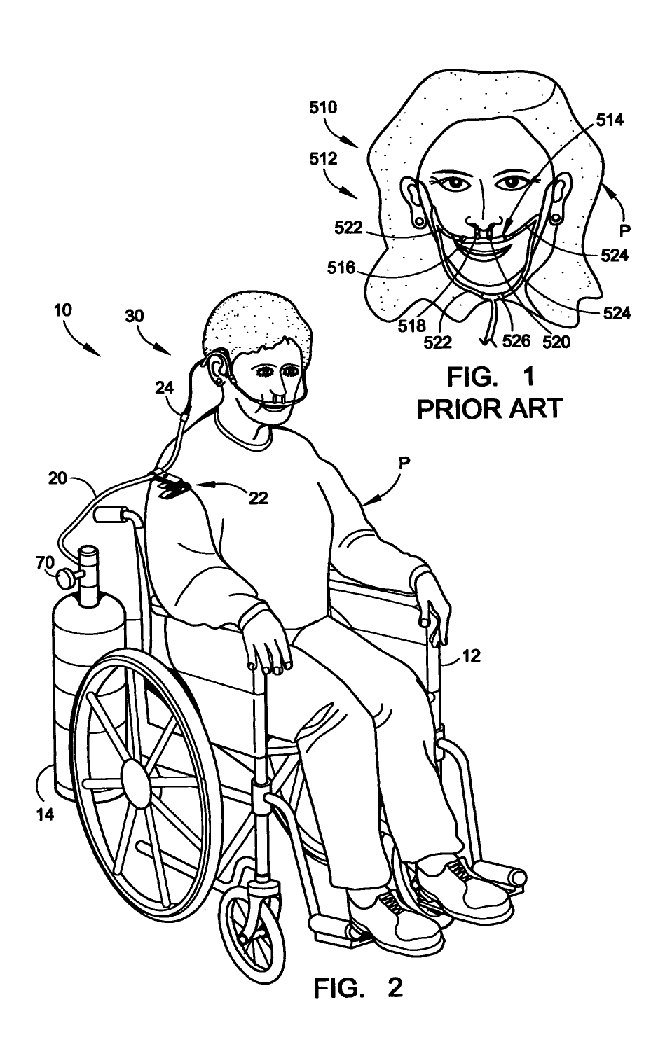

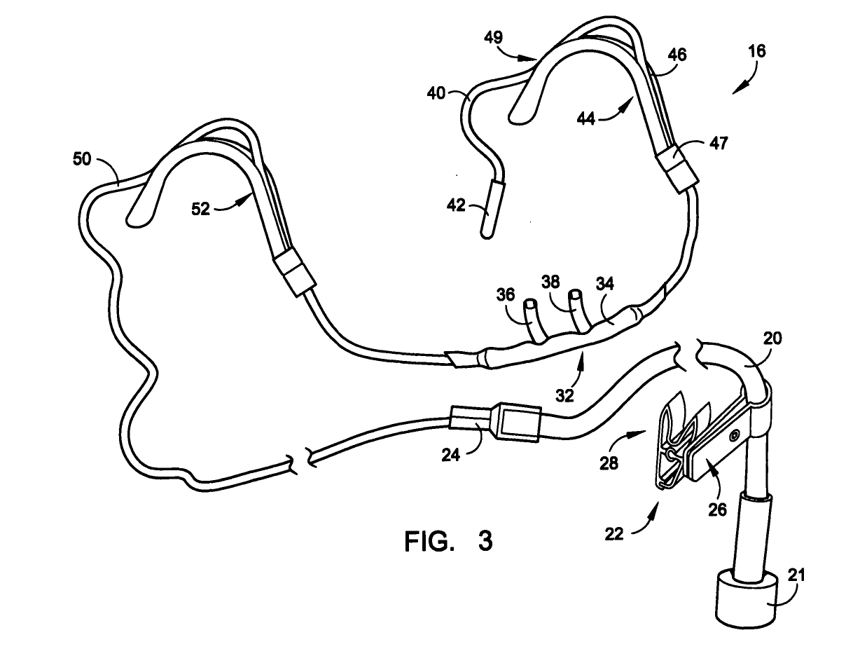

Referring now to the drawings and more particularly to FIGS. 2-3, there is shown a cannula system 10 that is constructed in accordance with the present invention. The cannula system 10 is illustrated being utilized by a patient P who is sitting in a wheelchair 12 that is adapted to transport a source of oxygen or air shown generally at 14.

Considering now the cannula system 10 in greater detail, the cannula system 10, generally comprises a nasal cannula assembly 16 and a flexible tube member 20 that cooperate to facilitate the delivery of air to the lung of the patient P. The flexible tube member 20 is held within a securing arrangement 22 that permits the tube member 20 to be secured to the shirt of the patient P or to a bottom bed sheet of a bed should the patient be confined to a bed.

In order to provide the nasal cannula assembly 16 with a supply of air, the flexible tube member 20 is connected at ones of its ends to a universal connector 21 that permits the flexible tube member 20 to be attached to the source of oxygen 14. A reduction connector 24 is connected to the other end of the flexible tube member 20 that permits the flexible tube member 20 to be attached to the nasal cannula assembly 16. As will be explained hereinafter in greater detail, the nasal cannula assembly 16 is adapted to be comfortably supported from the ears of the patient P.

Considering now the nasal cannula assembly 16 in greater detail with reference to FIG. 3, the nasal cannula assembly 16 generally includes a nasal delivery tube or cannula nosepiece 32, a pair of adjustment or extension tubes 40 and 50 respectively, and a pair of ear pieces 44 and 52 respectively. The extension tubes 40 and 50 are slidably mounted to the ear pieces 44 and 52 respectively in such a manner to facilitate supporting the cannula nosepiece 32 from the ear pieces 44 and 52 and to help facilitate adjusting the distance between the nosepiece 32 and individual ones of the ear pieces 44 and 52 so the cannula nosepiece 32 can be properly position relative to the nostrils of the patient P.

Considering the nasal cannula assembly 16 in still greater detail, the cannula nosepiece 32 includes a hollow body member 34 with two upstanding nose prongs or nasal extension tubes 36 and 38. The nose prongs 34 and 36 are adapted to be placed in the nasal cavities of the patient P as best seen in FIG. 2. In this manner, when the cannula nosepiece 32 is supported in the nasal cavities of the patient P it facilitates the delivery of oxygen to the lungs of the patient P in a comfortable and convenient manner. The nose prongs 34 and 36 are spaced apart from one another and have a sufficient length so as not to be dislodged from the nostrils of the patient. In this regard, the nose prongs 34 and 36 may be adjusted by cutting or trimming their ends with a pair of scissors (not shown) to a proper length to be comfortable to an individual patient, such as the patient P.

The hollow body member 34 is connected at its distal end in an airtight manner to the extension tube 40, which is plugged at its distal end with a plastic stop 42. The extension tube 40 is supported spaced from the ear of the patient P by the earpiece 44. As best seen in FIG. 18, the earpiece 44 has a recessed channel 46 and a pair of tube guide 47 and 48 respectively. In this regard, the extension tube 40 is sufficiently long to pass by its distal end through the front tube guide 47 and then along the channel 46 exiting at a rear portion 49 of the earpiece 44 through the rear tube guide 48. The channel 46 is sufficiently narrow to hold or capture the extension tube 40 against the ear piece 44, but not so narrows as to prevent the extension tube 40 from being pulled under a directed force through the channel 46 to a desired position. In this manner the patient P or a health care provider (not shown) may adjusts the distance between the nasal cannula 32 and the ear piece 44 to help position the nasal cannula 32 in proper position relative to the nostrils of the patient P for delivery of fluids to the lungs of the patient P.

From the foregoing those skilled in the art will understand that the stop 42 is attached to the distal end of the extension tube 40 after the tube 40 has been attached to the earpiece 44. Those skilled in the art will further understand that extension tube 40 is supported from the ear piece 44 in such a manner that the tube 40 does not rub against and irritate the ear of the patient P and that the extension tube 40 and ear piece 44 cooperate with one another to support the nosepiece 32 much in the same way as glass lens are supported but without the necessity of utilizing the bridge of the nose since the nosepiece 32 is substantially lighter in weight than that of glasses. This arrangement therefore eliminates the necessity of looping tubes around the ears of the user and along the jaw area and chin area thereby allowing the nosepiece 32 to be worn and supported in a very comfortable manner from the ears of the patient P.

As best seen in FIG. 3, the hollow body member 34 is coupled at its proximate end to the other extension tube 50 whose distal end is coupled in an airtight manner to the reduction connector 24. In this manner, a fluid or air path is established between the oxygen source 14 and the nasal cannula assembly 16 when the extension tube 50 is interconnected to the reduction connector 24. In the preferred embodiment of the present invention, the reduction connector 24 has been described as being attached to the flexible tube member 20. It should be understood however, by those skilled in the art, that the reduction connector 24 could be attached to the end of the extension tube 50 as part of the nasal cannula assembly 16.

The extension tube 50 is slidably mounted to the earpiece 52 and cooperates with the earpiece 52 to further facilitate supporting the cannula nosepiece 32. As the earpiece 52 is similar in construction to the earpiece 44, earpiece 52 will not be described hereinafter in greater detail. In a similar nature, as the manner of adjusting the position of the cannula nosepiece 32 relative to the ear piece 52 is substantially similar as the distance adjustment between the earpiece 44 and the cannula nosepiece 32, no further disclosure relative to adjustment is necessary.

From the foregoing it should be understood, that the nasal cannula assembly 16 is light in weight, is easily attached to an oxygen source, such as the oxygen source 14, utilizing a single tube path, and can be easily adjusted to fit and be supported from the ears of any patient, such as the patient P. Another important feature of the preferred embodiment of the present invention is that the nasal cannula assembly 16 is compact, simple in construction and does not necessitate the utilization of looping tubes around the ears of the user and along the jaw area and chin area thereby allowing the cannula 32 to be worn and support in a very comfortable manner.

Considering now the novel method of using the cannula system 10 with reference to FIGS. 2-4, the patient P first inserts the nostril prongs 34 and 36 of the nosepiece 32 into his or her nostrils to make certain that their overall length is a comfortable fit within the nostrils. If not, the tips of the prongs 34 and 36 may be cut to adjust them to a sufficient length to facilitate the comfort of the patient. P. Next as best seen in FIG. 2, the patient P hooks the earpieces 44 and 52 around his or her ears in the same manner as if putting on a pair of glasses allowing the nasal cannula nosepiece 32 to be freely supported by the ears of the patient P. If the nose piece 32 is not properly supported within the nostrils of the patient P, the patient P or a healthcare provider may grasps the extension tube 40 between his or her thumb and forefinger at about its exit point from the rear guide 48 of the ear piece 44 and the earpiece 44 between the thumb and forefinger of his or her other hand and then gently pull the extension tube 40 rearward to cause the cannula nosepiece 32 to move closer to the ear piece 44. Conversely, if the cannula nosepiece 32 needs to be adjusted so that it is moved further away from the earpiece 44, the patient may grasps the extension tube 40 between his or her thumb and forefinger at about its entry point into the front guide 47 and the ear piece 44 between the thumb and forefinger of his or her other hand and then gently pull the tube 40 forward to move the cannula nosepiece 32 further away from the earpiece 44. This procedure is repeated until the cannula nosepiece 32 is positioned at a proper distance from earpiece 44.

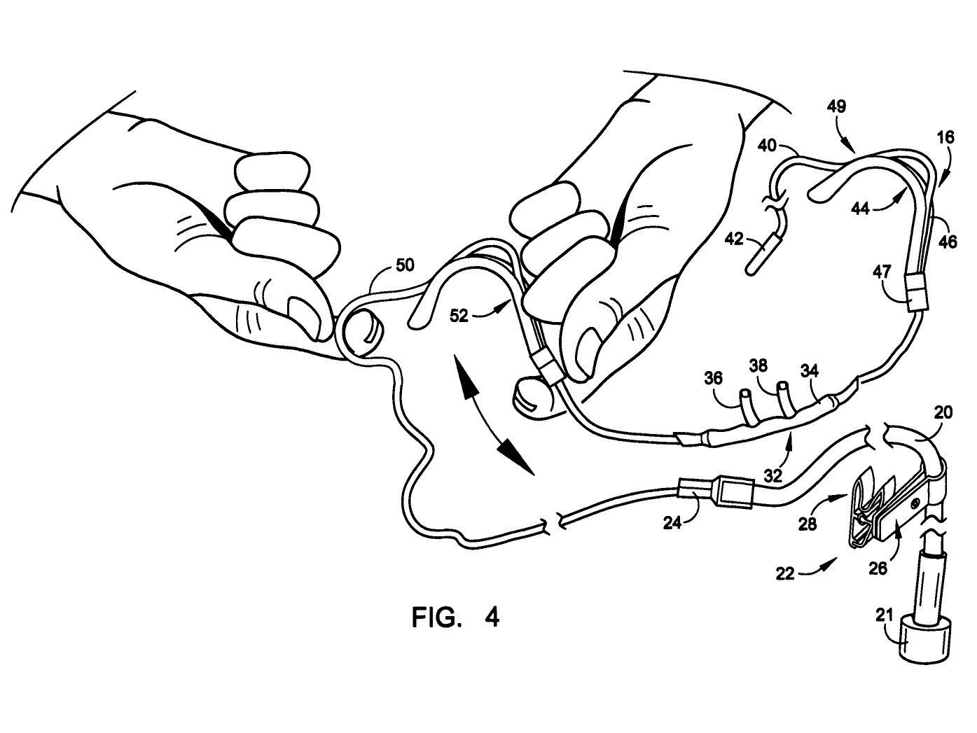

Next as best seen in FIG. 4, the above described procedure is repeated by the patient grasping the extension tube 50 between his or her thumb and forefinger at about its exit point from the rear guide of the ear piece 52 and the ear piece 52 between the thumb and forefinger of his or her other hand and then gently pulls the extension tube 50 rearward to move the nosepiece 32 closer to the earpiece 52. Conversely, if the nosepiece 32 needs to be adjusted so that it is moved further from the earpiece 52, the patient P may grasps the extension tube 50 between his or her thumb and forefinger at about its entry point into the front guide and the ear piece 52 between the thumb and forefinger of his or her other hand and then gently pulls the tube 50 forward to move the nosepiece 32 further away from the earpiece 52. This procedure is repeated until the nosepiece 32 is positioned at a proper distance from ea piece 52, thereby allowing the nose prongs 36 and 38 to be pulled up into the nostrils of the patient where they are disposed in a comfortable position and supported by the ear pieces 44 and 52 supported from the ears of the patient P.

In a final step, the patient couples the distal end of the extension tube 50 to the flexible tube 20, which was previously coupled to the source of air 14 as best seen in FIG. 2. The patient P may then turn on the supply of air using an actuation knob 70 allowing the free flow of oxygen to the nosepiece 32 for distribution into the lungs of the patient P.

Considering now the securing arrangement 22 in greater detail with reference to FIGS. 3-16, the securing arrangement 22 generally includes a flexible plastic strap 26 and a plastic clip 28. The flexible strap 26 is adapted to be secured in a friction tight fit around the tube member 20 without pinching or closing off the flow of fluids within the tube member 20, and is further adapted to be coupled to the plastic clip 28 for holding the clip 28 in a stationary position relative to the strap 26. From the foregoing it should be understood by those skilled in the art that the securing arrangement 22 is composed on two plastic parts that are coupled together without the use of any metallic parts, which allows the arrangement 22 to be easily and quickly assembled at a relatively low cost.

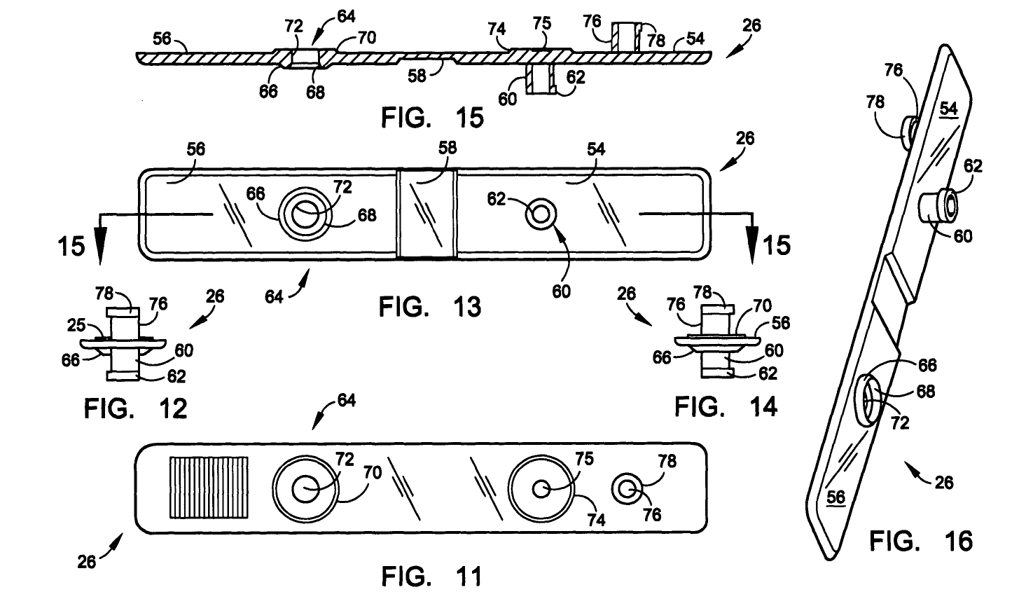

Considering now the flexible plastic strap 26 in greater detail with reference to FIGS. 11-16, the strap 26 is generally rectangular in shape having right side portion 54 and a left side portion 56 which are separated from one another by centrally disposed cutout section 58. The right side portion 54 and the left side portion have an overall thickness that is substantially greater the thickness of the cutout section 58. In this regard, the thickness of the cutout section 58 is sufficient thin to allow the plastic to wrap around the tube member 20 as best seen in FIGS. 3-4, without pinching the tube 20 so that it is incapable of a sustained flow of fluid under pressure from the air source 14.

In order to facilitate securing the strap 26 around the tube 20, the right side portion 54 includes an upstanding post 60 having a flange 62 at its distal end. The flange 62 is slightly offset from the post 60 and is constructed to be received within a catch 64 that extends through the left side portion 56. More particularly, the catch 64 has a slight boss 66 with a tapered opening 68 on its one side in the same plane as the post 60 and another slight boss 70 with a cylindrical opening 72 on its side opposite to the post 60. With this construction, the flange 62 slides within the tapered opening 68 and passes through the opening 72 allowing the post 60 to snap into locking engagement within the boss 70. As best seen in FIG. 12, the post 60 extends a significant distance beyond the upper surface of the boss 70. This is an important feature, as post 60 acts as an anchor point for the clip 28 as will be explained hereinafter in greater detail.

As best seen in FIGS. 11 and 15, the right side portion of the strap 26 includes another slight boss 74 having a centrally disposed opening 75 that is disposed opposite the post 60. The boss 74 functions as a finger receiving area for facilitating pressing post 60 into hole 72 when the strap 26 is attached to tube 20 as best seen in FIGS. 3-4. The right side portion 54 of the strap 26 also includes another post 76 that is disposed slightly outward from the boss 74. The post 76 includes a flange 78 at its distal end that is slightly offset from the post 76 to facilitate capturing the post 76 in hole 92 of the clip 83 as best seen in FIG. 10.

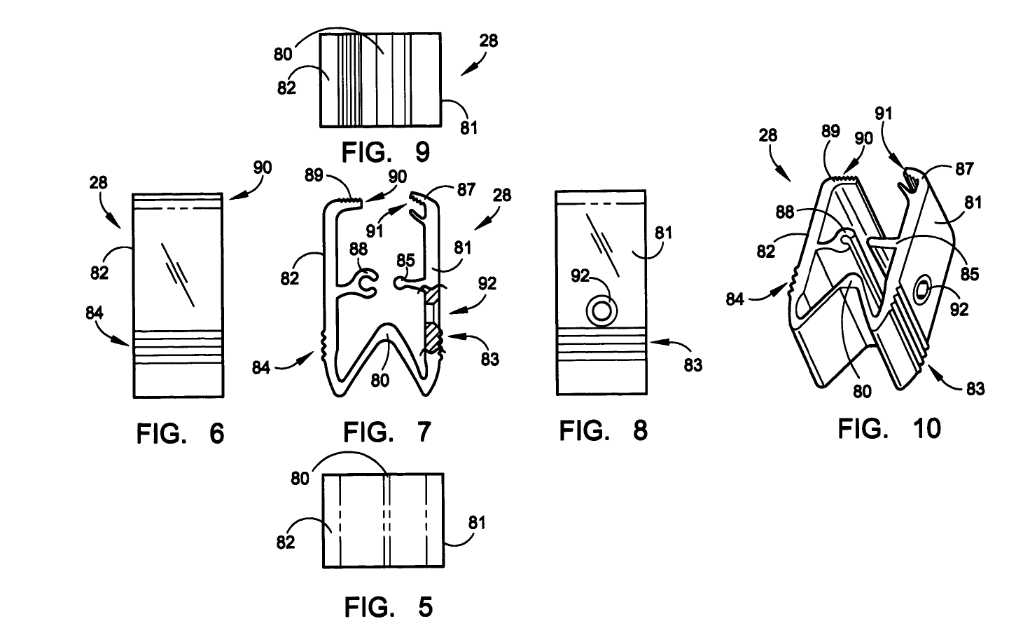

Considering now the plastic clip 28 in greater detail with reference to FIGS. 5-10, the plastic clip 28 includes a V-shaped pincher 80 that is centrally disposed and integrally connected between a right leg member 81 and a left leg member 82. The outer surface area of the right leg member 81 and the left leg member 82 each include a plurality of finely spaced apart finger engagable ridges indicated generally at 83 and 84 respectively. The ridges 83, 84 help keep the finger surfaces of the patient engaged with the upper surfaces of the leg members 81, 82 when the leg members 81, 82 are pinched toward one another at about the pincher 80.

As best seen in FIG. 7, the right leg member 81 includes a centrally disposed male member 85 which extends inward toward the center of clip 28 and a female member 87 which is disposed at the distal end of the right leg member 81 farthest from the pincher 80. The left leg member 82 includes a centrally disposed female member 88 which extends inward toward the center of the clip 28 and a male member 89 which is disposed at the distal end of the left leg member 82 farthest from the pincher 80. The male member 85 and the female member 88 are aligned so that when the right leg member 81 and the left leg member 82 are pinched toward one another the male member 85 and the female member 88 will come into locking engagement with one another. In a similar manner, the female member 87 and the male member 89 are aligned so that when the right leg member 81 and the left leg member 82 are pinched toward one another the female member 87 receives the male member 89.

In order to help facilitate the capture of a cloth material between the female member 87 and the male member 89 each of the members 87 and 89 include a plurality of ridges and valleys indicated generally at 90 and 91 respectively.

As best seen in FIGS. 7-8, the right leg member 81 of the clip 26 includes a tapered wall opening 92 that is dimensioned for receiving in locking engagement the post 60. In this regard, the flange 62 and post have a sufficient length to pass through the opening 92 and to be captured against the inner wall of the right leg member 81.

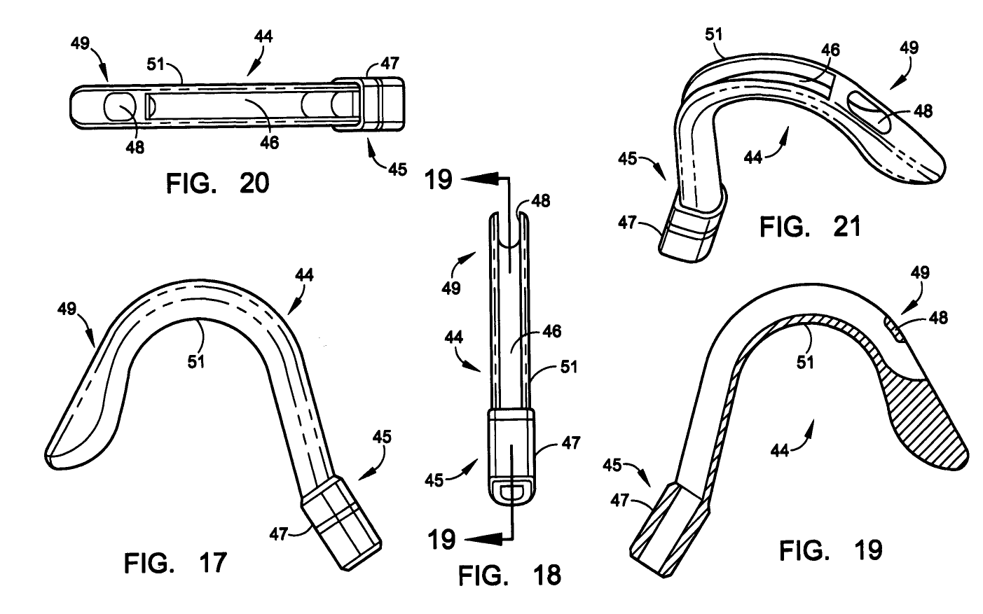

Considering now the earpiece 44 in greater detail with reference to FIGS. 17-21, the earpiece 44 and earpiece 52 are identical to one another and both are composed of a soft elastomeric material that is common to glassware earpieces. The earpiece 44 is a molded and includes a front part 45, which includes the front tube guide 47 and a rear part 49, which includes a curved rear channel section 51 which includes the channel 49 and exit guide 48.

While particular embodiments of the present invention have been disclosed, it is to be understood that various different modifications are possible and are contemplated within the true spirit and scope of the appended claims. For example, the ear piece support tube 40 described as having its distal end plugged with a removable plastic stop 42 could also be crimped or clamped at its distal end to provide an airtight seal preventing fluid from escaping from the distal end of the tube 40. As another example, the securing arrangement 22 is described as being constructed of all plastic parts could also have metal parts. Based on the foregoing, there is no intention, therefore, of limitations to the exact abstract or disclosure herein presented.

I claim:

1. A cannula system, comprising:
a nasal cannula for facilitating the delivery of fluids to the lungs of a user;
a pair of slider extension tubes coupled to said nasal cannula; and
a pair of ear pieces, each ear piece having a recessed tube channel for receiving slidingly therein an individual one of said pair of slider extension tubes to facilitate supporting from the ear piece said nasal cannula and to help facilitate adjusting the distance between the nasal cannula and individual ones of said pair of ear pieces to position
said nasal cannula in proper position relative to the nostrils of said user for the delivery of fluids to the lungs of the user.

2. A fluid delivery system according to claim 1, wherein said nasal cannula includes a pair of spaced apart nasal tips of sufficient length for insertion into the nostrils of the user.

3. A fluid delivery system according to claim 2, wherein said pair of spaced apart nasal tips have substantially smaller outer diameter than said nasal delivery tube.

4. A fluid delivery system according to claim 3, wherein said pair of spaced apart nasal tips are trimmable to custom fit the user.

5. A fluid delivery system according to claim 1, wherein each individual one of said ear pieces has disposed on its proximal end a guide for helping to facilitate guiding an individual one of said pair of extension tubes into a corresponding one of said recessed tube channels and to facilitate securing slidingly said individual one of said pair of extension tubes to said ear piece.

6. A fluid delivery system according to claim 5, wherein said nasal delivery tube includes a pair of spaced apart nasal tips of sufficient length for insertion into the nostrils of a user.

7. A fluid delivery system according to claim 6, wherein said pair of spaced apart nasal tips have substantially smaller outer diameter than said nasal delivery tube.

8. A fluid delivery system according to claim 7, wherein said pair of spaced apart nasal tips are trimmable to custom fit the nostril depths of the user.

9. A fluid delivery system according to claim 11, wherein each individual one of said ear piece recessed tube channels is sufficiently long to capture an individual one of said pair of extension tubes at two capture points and is sufficiently narrow at about a distal end thereof to fixedly secure said individual one of said pair of extension tubes within said channel at one of the two capture points to help facilitate supporting said nasal cannula substantially below the nose of the user and in close proximity to the nostrils of the user.

10. A fluid delivery system according to claim 1, further comprising:
a section of fluid delivery tubing coupled to said at a distal end of the other one of said pair of slider extension tubes and having a fluid source connector disposed at its distal end to help facilitate the delivery of fluids to the lungs of the user.

11. A fluid delivery system according to claim 10, further comprising:
a securing clip mounted to said section of fluid delivery tubing to help secure the fluid delivery tubing in a fixed position relative to the user.

12. A fluid delivery system according to claim 1, further comprising:
a securing clip mounted to the other one of said pair of slider extension tubes to help secure the other one of said pair of slider extension tubes in a fixed position relative to the user.

13. A fluid delivery system according to claim 1, wherein said fluid source is a source of air.

14. A fluid delivery system according to claim 1, wherein said fluid source is a source of oxygen.

15. A fluid delivery system according to claim 1, wherein said fluid source is a gas mixture source to help facilitate user breathing.

16. A method of delivery fluid to a user, comprising the steps of:
providing a nasal cannula having nostril tips;
providing a pair of slider extension tubes coupled to said nasal cannula;
providing a pair of ear pieces, each ear piece having a recessed tube channel;
sliding an individual one of said pair of slider extension tubes through one of the recessed tube channels to facilitate supporting from the ear piece said nasal cannula and to help facilitate adjusting the distance between the nasal cannula and an individual one of said pair of ear pieces to position said nasal cannula in proper position relative to the nostrils of said user for the delivery of fluids to the lungs of the user;
sliding another individual one of said pair of slider extension tubes through the other one of the recessed tube channels to facilitate supporting from the other ear piece said nasal cannula and to help facilitate adjusting the distance between the nasal cannula and another individual one of said pair of ear pieces to position said nasal cannula in proper position relative to the nostrils of said user for the delivery of fluids to the lungs of the user; and
placing a stop at a distal end of one of said pair of slider extension tubes and wherein the other one of said pair of slider extension tubes has a distal end adapted to be coupled to a fluid source.

17. The method of delivery fluid to a user according to claim 16, further comprising the steps of:
placing one of said ear pieces over one ear of the user to support therefrom one end of said nasal delivery tube;
placing another one of said ear pieces over another ear of the user to support therefrom an opposite end of said nasal delivery tube;
inserting respective ones of said nostril tips into corresponding ones of the nostrils of the user; and
sliding respective ones of said extension tubes within respective ones of said recessed channels to further adjust said nasal cannula to a user desired position where said nostril tips comfortable rest within the nostrils of the user.

18. The method of delivery fluid to a user according to claim 17, wherein said step of inserting includes trimming the length of respective ones of said nostril tips to custom fit them to the nostrils of the user.

19. An oxygen delivery system, comprising:
a nasal cannula having a pair of nasal prongs and a pair of extension tubes is plugged at a distal end thereof with a stop and is adapted to be coupled at a proximate end thereof to a supply of air;
a pair of ear pieces with recessed channels for helping to space said pair of extension tubes from the ears of a user; and
said pair of extension tubes cooperating with said pair of ear piece to facilitate positioning said pair of extension tubes within said respective ones of the recessed channels to support said nasal cannula from the ears of the user with said nasal prongs inserted into the nasal cavities of the user.

20. The cannula system according to claim 1, further comprising:
a stop disposed at a distal end of one of said pair of slider extension tubes and wherein the other one of said pair of slider extension tubes has a distal end adapted to be coupled to a fluid source.
